SEQUENCE LISTING (8 sequences)

SEQ ID NO: 1 - DNA, 2826 bp, Homo sapiens
ttagcttcctgctgcagacccgccggccgattctcctctgctctccacgtctcatgaagccgctggtcgtgttcgtcctcggcggccccggcgcgggcaaggggacccagtgcgcccgcatcgtcgagaaatatggctacacacacctttctgcaggagagctgcttcgtgatgaaaggaagaacccagattcacagtatggtgaacttattgaaaagtacattaaagaaggaaagattgtaccagttgagataaccatcagtttattaaagagggaaatggatcagacaatggctgccaatgctcagaagaataaattcttgattgatgggtttccaagaaatcaagacaaccttcaaggatggaacaagaccatggatgggaaggcagatgtatctttcgttctcttttttgactgtaataatgagatttgtattgaacgatgtcttgagaggggaaagagtagtggtaggagtgatgacaacagagagagcttggaaaagagaattcagacctaccttcagtcaacaaagccaattattgacttatatgaagaaatggggaaagtcaagaaaatagatgcttctaaatctgttgatgaagtttttgatgaagttgtgcagatttttgacaaggaaggctaattctaaacctgaaagcatccttgaaatcatgcttgaatattgctttgatagctgctatcatgacccctttttaaggcaattctaatctttcataactacatctcaattagtggctggaaagtacatggtaaaacaaagtaaatttttttatgttcttttttttggtcacaggagtagacagtgaattcaggtttaacttcaccttagttatggtgctcaccaaacgaagggtatcagctatttttttttaaattcaaaaagaatatcccttttatagtttgtgccttctgtgagcaaaactttttagtacgcgtatatatccctctagtaatcacaacattttaggatttagggatacccgcttcctctttttcttgcaagttttaaatttccaaccttaagtgaatttgtggaccaaatttcaaaggaactttttgtgtagtcagttcttgcacaatgtgtttggtaaacaaactcaaaatggattcttaggagcattttagtgtttattaaataactgaccatttgctgtagaaagatgagaaaacttaagctttgttttactacaacttgtacaaagttgtatgacagggcatatctttgcttccaagatttgggttgggggcactaggggttcagagcctggcagaattgtcagctttagtctgacataatctaagggtatggggcaaggatcacatctaatgcttgtgttccttatactctattatatagtgttattcatgattcagctgatcttaacaaaattcgtagcagtggaaccttgaaatgcatgtggctagatttatgctaaaatgattctcagttagcattttagtaacacttcaaaggtttttttttgtttgttttctagacttaataaaagcttaggattaattagaagaagcaatctagttaaatttcccatttgtattttattttcttgaatacttttttcatagttatttgtttaaaaagatttaaaaatcattgcactttggtcagaaaaataataaatatatcttataaatgtttgattcccttccttgctatttttattcagtagatttttgtttggcatcatgttgaagcaccgaaagataaatgatttttaaaaggctatagagtccaaaggaatattcttttacaccaaattcttcctttaaaaatctctgaggaatttgttttcgccttacttttttttcttctgtcacaatgctaagtggtatcccgaggttcttaatatgagatttaaaatcttaaaatgtttcttattttcagcacttacatcatttggtacaagggtcaaatagggcaaataattttgtctttgtataatagatttgatatttaaagtcactggaaataggacaagttaaggatgtttttatattttaatagaatcatttatttctatgtgttatgaaattcacttaatgataaatttttcaacatacttgccattagaaaacaaagtattgctaagtactataacatattggccactaaaattcatattgagattatcttggtttcttggaagagataggaatgagttcttatctagtgttgcaggccagcaaatacagaggtggtttgatcaaacagctctagtatgaagcaagagtaaggactaaggtttcgagagcattcctactcacataagtgaagaaatctgtcagataggaatctaaatatttatagtgagattgtgaaagcaccttaaagttttgaagaagactgatgagactaggtgctttgcttcctttcatcaggtatctttctgtggcatttgagaacagaaaccaagaaacatggtaattactaaattatgaggctttgctttttgtttgcttttaagtagaaaaacatgttggcaacattgagttttggagttgattgagataatatgacttaactagttttgtcattccatttgttaaagatacagtcaccaagaatgttttgagttttttgaaagaccccaatttaagccttgcttatttttaaattatttccattcagtgatgttggatgtatatcagttatttagtaaataatctcaataaattttgtgctgtggcctttgctaaaaaaaaaaaaaaaaaaaaaaaaaaaaaaaaaaaaaaaaaagaattccaccacactggcggccgctcgagcatgcatctagagggcctat

SEQ ID NO: 2 - protein, 196 aa, Homo sapiens [sequence omitted]

SEQ ID NO: 3 - protein, 182 aa, Sus scrofa
MRPKVVFVLGGPGAGKGTQCARIVEKYGYTHLSAGELLRDERKNPDSQYGELIEKYIKDGKIVPVEITISLLRREMDQTMAANAQKNKFLIDGFPRNQDNLQGWNKTMDGKADVSFVLFFDCNNEICIERCLERGKSSGRSDDNRESLEKRIQTYLQSTKPIIDLYEEMGKVKKIDASKSVD

SEQ ID NO: 4 - protein, 29 aa, Homo sapiens
GGPGSGKGTQCEKIVQKYGYTHLSTGDLL

SEQ ID NO: 5 - DNA, 138 bp, Homo sapiens
ctccctcagcgtccggccgaggcgcggtgtatgctgagccgctgccgcagcgggctgctccacgtcctgggccttagcttcctgctgcagacccgccggccgattctcctctgctctccacgtctcatgaagccgctg

SEQ ID NO: 6 - DNA, 64 bp, Homo sapiens
tagcttcctgctgcagacccgccggccgattctcctctgctctccacgtctcatgaagccgctg

SEQ ID NO: 7 - protein, 36 aa, Homo sapiens
MLSRCRSGLLHVLGLSFLLQTRRPILLCSPRLMKPL

SEQ ID NO: 8 - DNA, 173 bp, Homo sapiens
ggcacgaggccccgcgccgcgccggccgctgtcagctccctcagcgtccggccgaggcgcggtgtatgctgagccgctgccgcagccggctgctccacgtcctgggccttagcttcctgctgcagacccgccggccgattctcctctgctctccacgtctcatgaagccgctg

(19) United States
(12) Patent Application Publication
(10) Pub. No.: US 2005/0100998 A1
(43) Pub. Date: May 12, 2005

(54) Human cytidine monophosphate (cmp) kinase cdna

(76) Inventors: Stephen M Prescott, Salt Lake City, UT (US); A. Terrece Pearman, Salt Lake City, UT (US); Hugo C Castro-Faria-Neto, Salt Lake City, UT (US); Diana M Stafforini, Salt Lake City, UT (US); Thomas A. McIntyre, Salt Lake City, UT (US)

Correspondence Address: MADSON & METCALF;GATEWAY TOWER WEST, SUITE 900, 15 WEST SOUTH TEMPLE, SALT LAKE CITY, UT, 84101, US

(21) Appl. No.: 10/362,332

(22) PCT Filed: Aug. 15, 2001
(86) PCT No.: PCT/US01/25567

Related U.S. Application Data
(60) Provisional application No. 60/226,614, filed on Aug. 21, 2000.

(51) Int. Cl. C12Q 1/68; C07H 21/04; C12N 9/12; C12P 21/02; C12N 5/06

(52) U.S. Cl. 435/194; 435/69.1; 435/320.1; 435/325; 435/6; 536/23.2

(57) ABSTRACT

The present invention provides the cDNA for a novel nucleoside/nucleotide monophosphate kinase cloned from a human macrophage cDNA library. This kinase is able to phosphorylate UMP and utilize multiple phosphate donors, but has demonstrated a preference for transferring a phosphate from ATP or UTP to cytidine monophosphate (CTP). The kinase is thus referred to as cytidine monophosphate (CMP) kinase. This kinase is shown to be a Mg2+ dependent nucleotide kinase which codes for two ubiquitously transcribed mRNA splice products of 3.2 and 2.0 kb, and has been mapped to chromosome 1, region p32-34.1, a region known to contain other nucleotide modifying enzyme genes.

### 1. RELATED APPLICATIONS

This application is related to and claims the benefit of U.S. Provisional Application Ser. No. 60/226,614 of Stephen M. Prescott, Terrece A. Pearman, Hugo C. Castro-Faria-Neto, Diana M. Stafforini, Guy A. Zimmerman, and Thomas M. McIntyre, filed Aug. 21, 2000 and entitled “Human Cytidine Monophosphate (CMP) Kinase cDNA,” which is incorporated herein by this reference.

### 2. FIELD OF THE INVENTION

The present invention relates to nucleoside/nucleotide modifying enzymes. More specifically, the present invention relates to a novel ubiquitously expressed human nucleoside monophosphate kinase that phosphorylates CNT and UMP. This novel kinase is capable of utilizing multiple phosphate donors. Its genomic location suggests that it may prove important in investigations of disease and studies focusing on the improvement of nucleoside analog drug efficacy.

### 3. TECHNICAL BACKGROUND

Nucleotides and nucleosides are important molecules that participate in many vital cellular functions. Nucleotides serve as carriers of chemical energy, cellular signaling molecules, and building blocks of nucleic acids. See Alberts, et al, Molecular Biology of the Cell, 3d ed., (1994). Nucleotides also combine with other groups to form enzymes. Structrally, nucleotides are composed of a purine or pyzimidine base attached to a ribose or deoxyribose sugar with one or more phosphate groups joined to the sugar by ester linkages. Nucleotides lacking the phosphate groups are called nucleosides. Energy is stored in nucleotides as bond energy by enzymes that attach the phosphate groups to them in a reaction called phosphorylation. These enzymes are commonly known as “kinases,” an abbreviation of the term “phosphokinase.”

The energy stored in nucleotides through phosphorylation resides in the bond energy of the bond holding the phosphate group to the nucleotide, and is released when the phosphorylated compound is hydrolyzed, resulting in the release of free phosphoric acid Kinase mediated energy storage and transmission is very important to many fundamental cellular processes and reaction pathways.

Kinases are members of a group of nucleoside/nucleotide modifying enzymes that act on nucleotides and nucleosides. In addition to phosphorylation, these enzymes govern functions ranging from nucleotide synthesis to nucleotide degradation. Proper function of kinases is critical to cellular function.

One example of the increasingly apparent importance of nucleoside/nucleotide modifying enzymes such as kinases that catalyze reactions other than de novo nucleotide synthesis is the participation of such kinases in scavenger pathways. In this function, kinases convert nucleotides into phospholipids and other cellular metabolites. These enzymes are critical because improper lipid composition unfavorably changes membrane properties and function. One example, of this is seen in Escherichia coli (E. coli). When the E. coli CMP kinase is inactivated, severe phospholipid synthesis defects are observed. This abnormal membrane lipid content causes observable cold sensitivity in the bacterium. Fricke et al., J. Bacteriol. 177:517-523 (1995). Similarly, incorrect membrane phospholipid makeup may cause the hemolytic anemia associated with adenylate kinase deficiency. Matsuura et al., J. Biol. Chem. 264:10148-10155 (1989).

Another example of the importance of kinases involves pyrimidine nucleoside monophosphate (PNMK) kinase, an enzyme whose hyperactivity accompanies a pyrimidine 5′ nucleotidase deficiency and is also associated with hemolytic anemia (Lachant et al., 1986). In patients with this disorder, erythrocyte survival is decreased, and CMP and UMP have been observed to accumulate. Further, adenylate kinase (AK-1) deficiency has been associated with congenital hemolytic anemia that is sometimes accompanied by mental or psychomotor retardation. (Szeinberg et al., 1969; Miwa et al., 1983; Boivin et al., 1971; Toren et al., 1994).

Without being bound to any one theory, these examples appear to point to the conclusion that nucleotide levels are critical to appropriate erythrocyte shape, function, and survival. Yet other studies suggest that these levels may also influence the behavior of other cells, including neurons. As a result, the provision and characteizaon of novel nucleotide/nucleoside kinases that influence cellular nuclotide/nucleoside levels would be a significant improvement in the art.

Nucleoside/nucleotide modifying enzymes are also critical to the pharmacological uses of nucleoside analogs. Nucleoside analogs are used in many treatment applications, including cancer treatments, HIV therapies, and battling viruses. Furman et al., Proc. Natl. Acad. Sci. USA 83:8333-8337 (1986); Cihar & Chen, Mol. Pharmacol. 50:1502-1510 (1996). In order to function, however, nucleoside analog drugs must first be activated by nucleoside kinases. As a result, the efficacy and usefulness of these drugs are hampered by a lack of understanding of how nucleotide kinases modify these compounds during activation.

From the foregoing, it will be appreciated that it would be an advancement in the art to provide a novel cDNA coding for a novel nucleoside/nucleotide modifying enzyme. Specifically, the provision of novel kinases and the elucidation of their chromosomal location and specific function could render possible better assessment of the half-lives of the nucleoside analog drugs noted above. It could further enable the extension of half-lives of these drugs. This would in turn increase drug efficacy and the potential benefits of such drugs to thousands of patients. It would thus be a clear improvement in the art to isolate a novel nucleoside/nucleotide modifying enzyme and characterize its associated products, activities, and expression patterns.

### 4. BRIEF SUMMARY OF THE INVENTION

The present invention relates to a novel human nucleoside monophosphate kinase cDNA isolated from a macrophage library. It further relates to the cloning of this kinase and the elucidation of its enzymatic activity, dependencies, and expression pattern. A sequence homology search identified human adenylate kinase-1 (AK-1) as the closest known match in the human genome.

In some embodiments, the instant invention comprises an isolated and purified nucleic acid molecule with a nucleotide sequence that encodes an ammo acid sequence at least 70%, 80%, 90%, or 95% identical to the amino acid sequence of SEQ ID NO: 2. In other embodiments, the sequence encodes the amino acid sequence of SEQ ID NO: 2. In yet other embodiments, the invention comprises an isolated nucleic acid molecule which hybridizes to the complement of the nucleic acid molecule of SEQ ID NO: 1 when incubated for one hour (30 ng nucleic acid molecule/ml Express Hyb) at 68° C., followed by washing twice for 15 min, 0.1% SDS/2×SSC at room temperatre and then by washing twice for 15 min, 0.1% SDS/0.2×SS at 68° C. Other embodiments of the invention include this nucleic acid molecule subcloned into a plasmid, a prokaryotic expression vector, or a eukaryotic expression vector. Still other embodiments of the invention include this nucleic acid molecule operably linked to a heterologous promoter. Other embodiments of this invention include this nucleic acid molecule stably or transiently incorporated into a prokaryotic or eukaryotic host cell.

The instant invention further relates to the catalytic activity of the UMP-CMP kinase. Specifically, it was found that the preferred substrates for this kinase are CMP and UMP. Since a three-fold greater reaction efficiency in the presence of CMP relative to UMP was observed, the novel kinase of the instant invention is alternatively referred to as CMP kinase. It was also discovered that CTP and UTP are the preferred phosphate donors for this enzyme, and that its function is Mg²⁺ dependent. A publication that described the results of cloning conducted after the isolation of this macrophage-derived cDNA contained data that affirms this result See Van Rompay et al., Mol. Pharmacol. 56:562-569 (1999).

The invention further relates to the expression patterns of the UMP-CMP kinase in humans. Investigation of the expression patterns of this UMP-CMP kinase revealed two mRNA products measuring 3.4 and 2.0 kb in immune tissues and cancer cell lines. The 2.0 kb mRNA has not been previously described.

The invention further relates to the elucidation of the sequence and size of the UMP-CMP kinase. An article published after the cloning of this cDNA disclosed a cDNA shown by sequence comparison to be derived from the same gene as that reported herein. Van Rompay et al., Mol. Pharmacol. 56:562-569 (1999). There is a conspicuous difference between the two clones, however. The sequence of the cDNA disclosed in the lae publication possesses 73 additional 5′ nucleotides not found in the macrophage-derived clone of the insant invention.

This extension disclosed in the later publication includes a putative translational start site located 96 nucleotides (coding for 32 putative amino acids) 5′ to the first ATG in the macrophage-derived cDNA of the intant invention. The inventors of the instant invention showed this additional 5′ sequence to be unnecessary for enzymatic activity in the gene product. The protein expr by the macrophage-derived cDNA of this invention has the same relative molecular mass as that produced by the extended cDNA of the later publication. These data indicate that the second ATG in the subsequently described clone is the functional start site.

These and other features of the present invention will become apparent upon reference to the accompanying figures and upon reading the following detailed description and appended claims.

### 5. SUMMARY OF THE DRAWINGS

A more particular description of the invention briefly described above will be rendered by reference to the appended figures. These figures only provide information concerning typical embodiments of the invention and are not therefore to be considered limiting of its scope.

FIG. 1 is the nucleotide sequence of the UMP-CMP kinase cDNA of the instant invention, including the 5′ and 3′ untranslated regions.

FIG. 2 is a comparison of the amino acid sequence of the UMP-CMP kinase and two closely homologous human enzymes.

FIG. 3 graphically illustrates the ability of the UMP-CMP kinase of the instant invention to convert approximately 6 times more CMP into CDP per mg. protein/min. than vector-transfected cells.

FIG. 4 graphically demonstrates the concentration-based dependence of UMP-CMP kinase activity on Mg²⁺.

FIG. 5 illustrates the preferred substrate (FIG. 5A) and preferred phosphate donor (FIG. 5B) of the UMP-CMP kinase.

FIG. 6 illustrates the tissue distribution of UMP-CMP kinase in immune tissues (FIG. 6A) and in cancer cell lines (FIG. 6B).

FIG. 7 shows the location of human UMP-CMP kinase on the short arm of chromosome 1, region p32-p34.1.

FIG. 8 shows a comparison of the 5′ end regions of an I.M.A.G.E. clone (cDNA_{—}469332), the previously-reported human cDNA for UMP-CMP kinase (accession no. AF070416), and the macrophagederived clone of the instant invention. Also shown is the predicted translation into protein sequence.

FIG. 9 shows the expression of CMP kinase cDNA clones and demonstrates that Met-33 is the preferred translational start site in vivo.

### 6. DETAILED DESCRIPTION OF THE INVENTION

The present invention relates to a ubiquitously expressed human nucleoside monophosphate kinase that phosphorylates CMP and UMP. It further relates to the cloning, enzymatic activity, and expression pattern of this kinase. The kinase's function as a nucleoside/nucleotide modifying enzyme makes it an attractive subject for research into disease and research into the improvement of nucleoside analog drug efficacy.

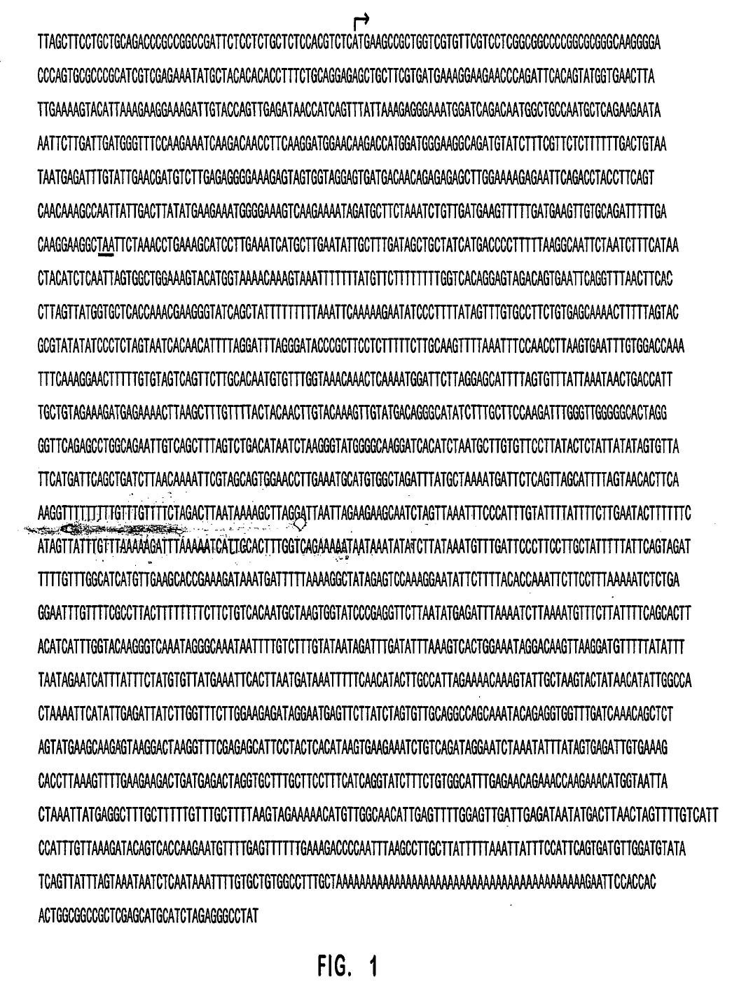

The original UMP-CMP kinase cDNA starting at nucleotide 74 of AF070416 was isolated from a human macrophage cDNA library. Tjoelker et al., Nature, 374: 549-552 (1995). It was further cloned into the BstXI sites of the mammalian expression vector pRc/CMV (Invitrogen). Sequencing of both sense and antisense DNA was accomplished with the use of an ABI automated sequencer. The nucleotide sequence, including 5′ and 3′ untranslated regions, is presented in SEQ ID NO: 1, and in FIG. 1. FIG. 1 shows the translational start site with an arrow and the translational stop site with underlining. A sequence comparison of the cDNA showed high homology with other nucleoside monophosphate kinases, with the highest homology being with the pig UMP-CMP kinase (Okajima et al., 1995). Within the human genome, the cDNA showed highest similarity to cytosolic adenylate kinase-1 (Matsuura et al., 1989).

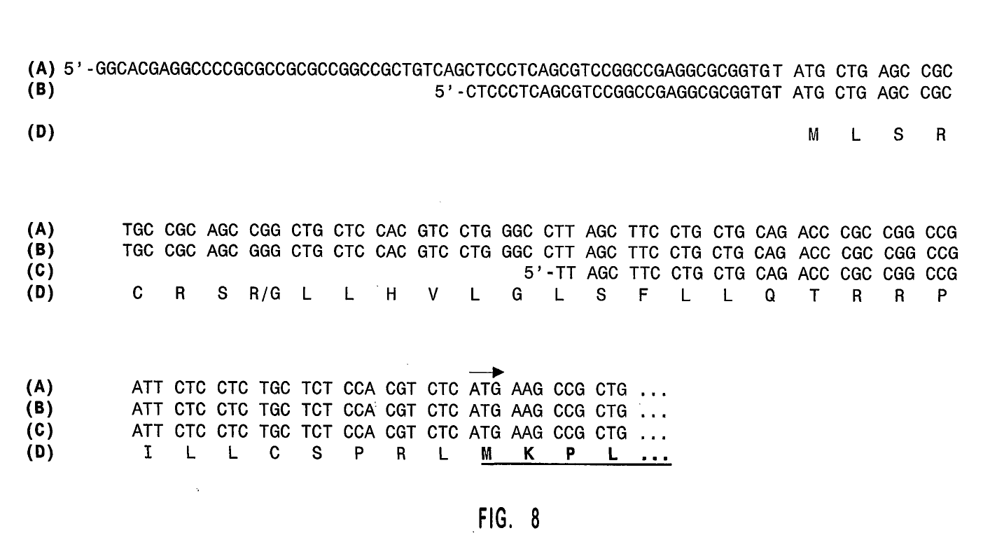

To compare the protein products synthesized by transfection of the entire UMP-CMP kinase cDNA versus the cDNA staring at position 74, an individual I.M.A.G.E. cDNA clone was obtained (cdna_id 469332) that was found to encode 65 bp of 5′UTR (FIG. 8), the entire coding region, and 1031 nt of the 3′UTR Products were amplified comprising nt 1-1561 and nt 74-1561 by amplification using Pfu DNA polymerase. The PCR products were cloned into the BstXI and Xba I sites of the mammalian expression vector pRc/CMV (Invitrogen). The sequence of each clone was verified by automated sequence analysis.

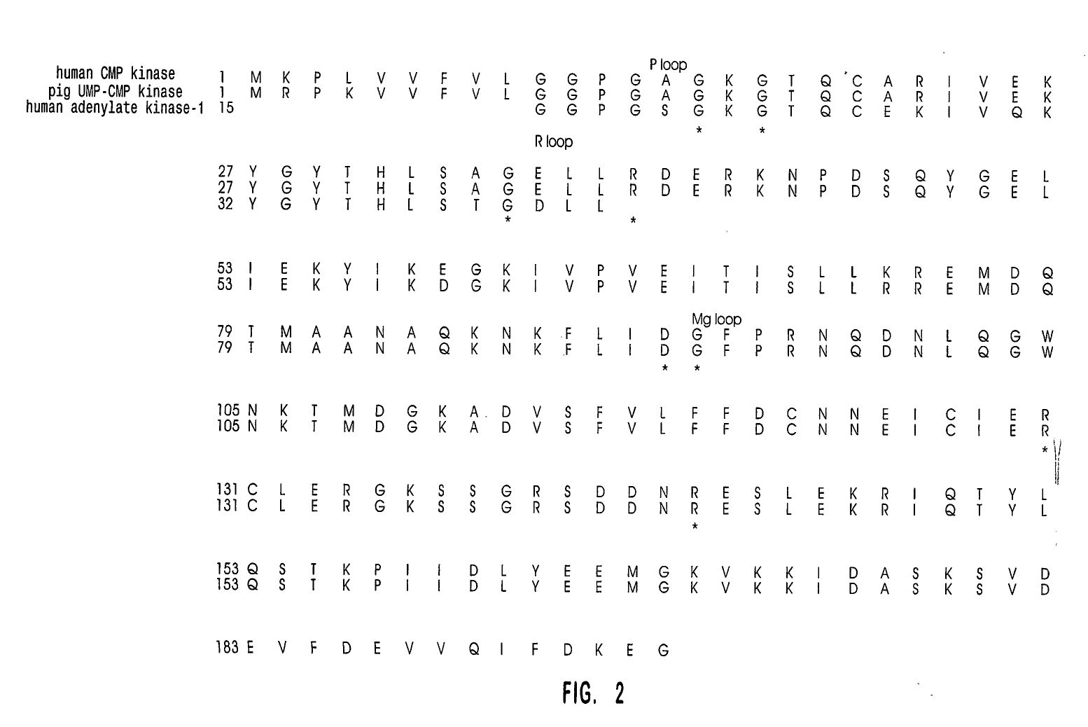

This cDNA codes for a predicted 196 amino-acid protein. Comparisons of the amino acid sequence (SEQ ID NO: 2) with other known sequences showed similarities with other nucleoside monophosphate (NMP) kinases, particularly within motifs known to confer functional properties. FIG. 2 is an amino acid sequence comparison of the human CMP kinase of the instant invention (SEQ ID NO: 2), pig UMP-CMP kinase (SEQ ID NO: 3), and human adenylate kinase-1 (SEQ ID NO: 4). Protein regions that share a significant similarity with other NW kinases include a phosphate-binding loop and an Mg²⁺ binding loop, as indicated in FIG. 2. FIG. 2 denotes highly-preserved residues thought to play a role in kinase function with an asterisk Bucurenci et al., J. Biol. Chem., 271:2856-2862 (1996). From these comparisons, it seemed reasonable that the active site, cation requirements, and general protein conformation might be similar to other NMP kinases.

The Basic Local Alignment Search Tool (BLAST) was then used to compare the cDNA sequence with other sequences submitted to the database. This search turned up an expressed sequence tag (EST) clone also referred to as UMP-CMP kinase (AF070416), which was cloned after the cloning of the cDNA of this invention. Van Rompay et al., Mol. Pharmacol., 56:562-569 (1999); (accession no. AF070416). It was determined that this EST clone was derived from the same gene as that of the macrophage-derived cDNA, but that the EST clone included an added 73 bases on the 5′ end as shown in FIG. 8, which compares the 5′ end regions of the two clones. FIG. 8 also contains the 5′ end of an I.M.A.G.E. clone (SEQ ID NO: 8) and the predicted translation of the sequence into protein. SEQ ID NO: 5 contains this 73-base section of the 5′ end of the cDNA of the EST clone described in Van Rompay et al. SEQ ID NO: 6 contains the 5′ end of the cDNA of the macrophage-derived clone of the instant invention. FIG. 8 shows the putative N-terminal 32 amino acids of the EST clone in plain type. These do not appear to exist in the translated product These 5′ nucleotides would add an additional putative translational start site to the sequence that if functional, would result in a 32-amino-acid N-terminal extension of the protein. FIG. 8 shows the amino acids that appear to actually be translated in bold type, and denotes the functional start site with an arrow. The 32 putative amino acids plus the four which are present in the functional protein are contained in SEQ ID NO: 7.

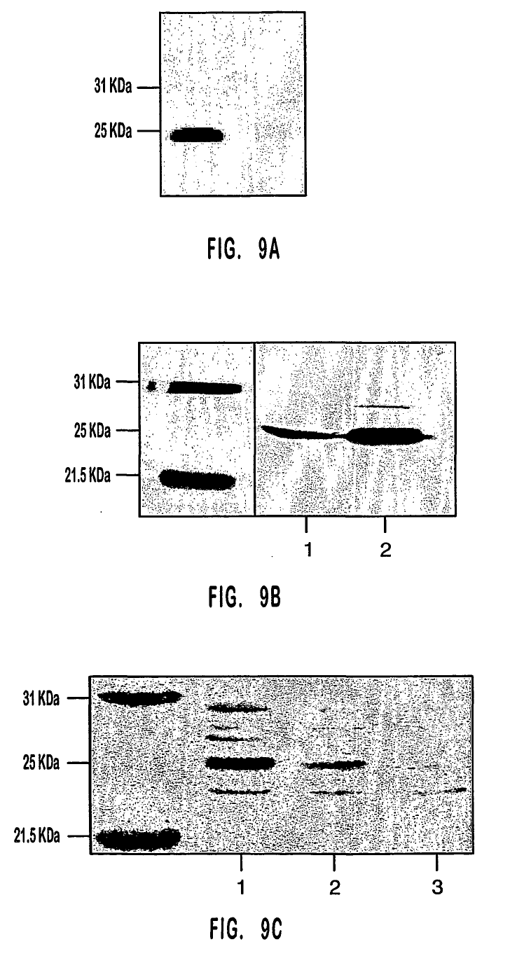

In order to assess whether the N-terminal extension is expressed, the macrophage-derived UMP-CMP kinase cDNA was expressed in HEK 293 cells transfected with UMP-CMP kinase cDNA cloned into a mammalian expression vector. The relative molecular mass of the protein expressed was determined by Western blot. This cDNA produced a 25 kDa protein, as seen in the left lane of FIG. 9A. The size of the product is slightly larger in size than that predicted from the raw sequence (22,222 Da), but similar which agrees with the size predicted for the 196-amino-acid sequence without the putative N-terminal peptide. The result is shown in FIG. 9A. The EST clone was reported to produce a protein of the same relative molecular mass. Van Rompay et al., Mol. Pharmacol., 56:562-569 (1999). The vector transfected cells shown in the right lane of FIG. 9A do not express this protein at detectable levels. This result is representative of three repetitions of the experiment. Identical results were obtained when COS-7 cells were similarly transfected.

Referring now to FIG. 9B, expression of the macrophage-derived UMP-CMP kinase in bacteria results in the production of a 25 kDa protein. Two aliquots (Lane 1: 90 ng; lane 2: 900 ng) of purified recombinant UMP-CMP kinase expressed in E. coli were subjected to SDS-PAGE and Western analysis. The results show that post-translational modifications do not account for the slightly higher molecular weight observed in transfected mammalian cells [25 kDa, panel (A)] versus the predicted molecular weight of 22,222 Da.

Referring now to FIG. 9C, cDNAs encoding UMP-CMP kinase staring at either Met-1 (lane 1) or Met-33 (lane 2) were transfected into HEK-293 cells as described in Methods. Empty vector (lane 3) served as a negative control. Extracts from transfected cells were subjected to Western analysis. Both constructs resulted in the production of UMP-CMP kinases that migrated with an apparent molecular weight of 25 kDa.

The protein expressed by the kinase transfected cells is similar in size to enzymes whose genes exhibit the closest homology, namely: pig UMP-CMP kinase (22.3 kDa, 196 amino acids) (Okajima et al., 1995), human adenylate kinase-1 (21 kDa, 194 amino acids) (Matsuura et al., 1989), and E. coli CMP kinase (24.6 kDa, 225 amino acids) (Bucurenci et al., J. Biol. Chem., 271:2856-2862 (1996)).

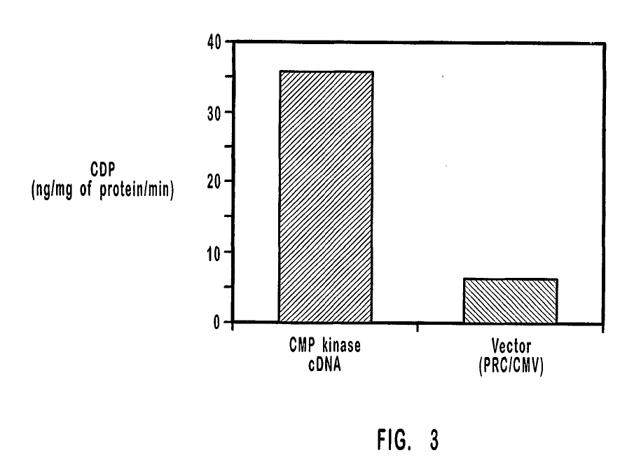

Tests were conducted on the enzyme produced by the cDNA. Based on its sequence similarity to the pig UMP-CMP kinase, tests were conducted to assess the enzyme's ability to phosphorylate CMP. It was demonstrated that lysates from cells transfected with the UMP-CMP kinase of this invention converted approximately 6 times more CMP into CDP per mg. protein/mi than vector-transfected cells, as shown graphically in FIG. 3. The amount of CDP formed was shown to increase with the amount of total cell lysate added to the reacton. The result shown in FIG. 3 is representative of seven repetitions of the experiment.

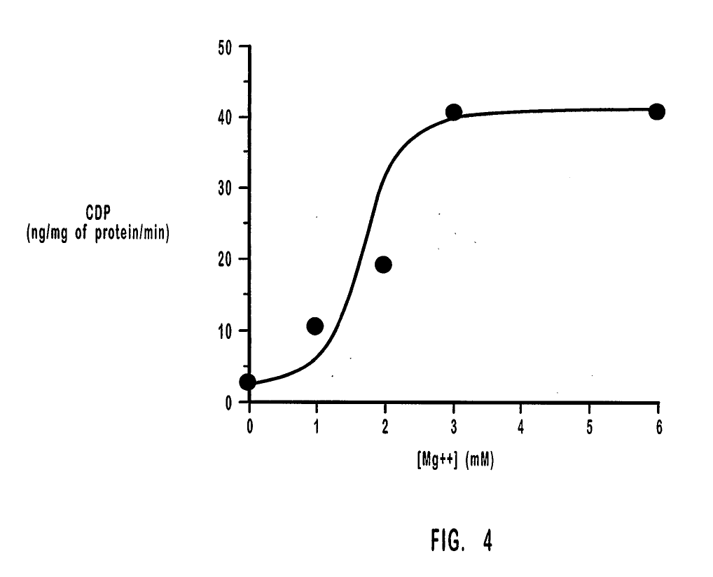

Since, as noted above, the UMP-CMP kinase has a region showing high similarity to Mg²⁺ loops found in other nucleoside kinases, the influence of Mg²⁺ concentration on the activity of the kinase was also investigated. Lysates from cells transfected with UMP-CMP kinase cDNA were added to solutions containing the shown Mg²⁺ concentrations. UMP-CMP kinase activity was then measured in the presence of varying concentrations of Mg²⁺. The result of this investigation was that Mg²⁺ increases UMP-CMP kinase activity in a concentration-dependent manner, as shown in FIG. 4. The absence of Mg²⁺ resulted in reduction of CDP production to the levels observed in vector-transfected cells, indicating an absolute requirement of the kinase for Mg²⁺. Maximal activity of the kinase was observed in the presence of 3 mM Mg²⁺. Without being limited to any particular theory, these findings prompted the conclusion that the putative Mg²⁺ binding loop in the predicted amino acid sequence of the kinase is functional.

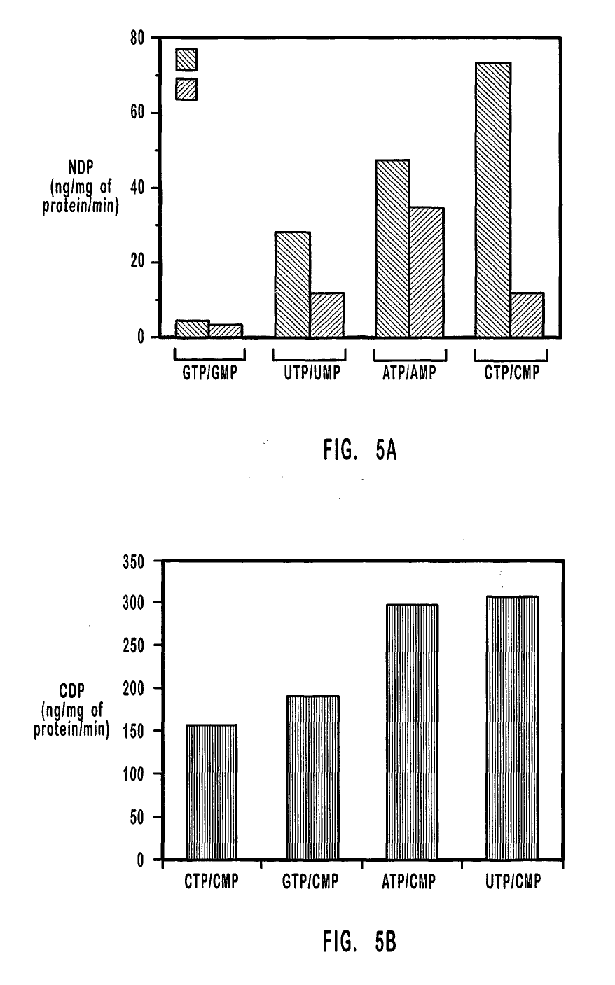

Since the macrophage-derived human UMP-CMP kinase shows similarities to the pig UMP-CMP kinase, it was predicted that the novel human enzyme would exhibit similar substrate preferences to those of the pig kinase. Since many NMP kinases phosphorylate multiple types of NMPs with varying relative kinetics, a variety of NMPs were tested as substrates. The preference of UMP-CMP kinase for transferring phosphate from either ATP or UTP to CMP was first measured. As shown in FIG. 5A, lysates of cells transfected with either UMP-CMP kinase or vector only were added to reaction mixes that included the indicated NTP and NMP. The amount of NMP converted to its respective NDP indicates the relative rates at which each NMP is phosphorylated by CMP kinase. The kinase was found to be unable to phosphorylate GMP, as noted in FIG. 5A. Additionally, it was shown to phosphorylate AMP only slightly, exhibiting levels only slightly above those of vector-transfected cells, as also shown in FIG. 5A. When UMP was offered as a substrate, however, a 2-fold increase in UDP formation was noted. See FIG. 5A. When CMP was made available as a substrate, a 6-7-fold increase in CDP production was exhibited. See FIG. 5A. This result is representative of 3 individual experiments.

The most preferred phosphate donor of the kinase was next investigated. As seen in FIG. 5B, lysates from cells transfected with UMP-CMP kinase were added to a reaction mix that included CMP as the substrate combined with the indicated NTP phosphate donor. The amount of CDP formed indicates the ability of each NTP to act as a phosphate donor. UTP and ATP were preferred as phosphate donors over CTP and GTP. Indeed, CTP appeared to be the least preferred acceptor, since it exhibited half of the efficiency of ATP or UTP. This result is representative of three independent experiments.

Van Rompay et al., claimed that CMP, UMP, and dCMP were efficiently phosphorylated by the kinase. Van Rompay et al., Mol. Pharmacol., 56:562-569 (1999). That study failed to show any difference in relative substrate preference between CMP and UMP. Variations in assay protocols (including reaction time differences and phosphate donor choice) may account for the differing results. A comparative kinetic analysis should show whether this is the case.

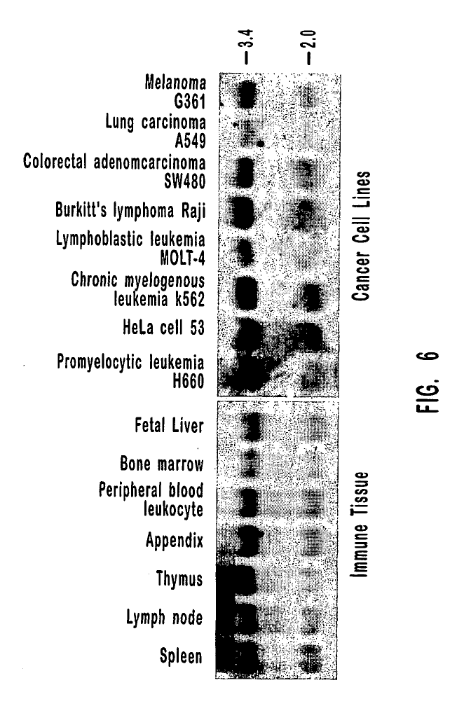

The tissue distribution of the kinase was next investigated. Though nucleoside kinases commonly show ubiquitous distribution, it was determined that this kinase should be evaluated to assess whether it was differentially expressed. Since the cDNA was cloned from a human macrophage library, immune tissues were assessed. See FIG. 6A for the results. CMP kinase mRNA levels were assessed by human immune multi-tissue Northern blot. Membranes were subsequently probed with human β-actin to ensure that equal RNA was loaded in each lane. Two different sizes of mRNA appeared (3.4 and 2.0 kb). Dual transcripts have also been described in human adenylate kinase-1, the gene with the highest similarity to the macrophage-derived cDNA within the human genome (Matsuura et al., 1989), and without being limited to any one theory, it was presumed that the UMP-CMP kinase may also have splice variants. The 3.4 kb form was the most prominent in every tissue, and neither form exhibited tissue-specific expression, as shown in FIG. 6A.

The possibility that UMP-CMP kinase was developmentally regulated as monocytes proceed toward the macrophage phenotype was also investigated. To assess this, human monocytes were isolated and cultered for 10 days as they differentiated into macrophages. RNA was isolated on the day of cell plating and then throughout the culture period. RT-PCR analysis showed no difference in CMP kinase mRNA levels throughout the process. In order to confirm this result, HL-60 cells were employed. These cells are monocytic, but differentiate into macrophage-like cells in the presence of phorbol-12-myristate-13-acetate (PMA) (Murao et al., 1983; Rovera et al., 1979), or into neutrophils in the presence of DMSO (Collins et al., 1975). PMA treatment induced phenotypic changes in the HL-60 cells that were similar to those observed when isolated monocytes differentiate into macrophages. These changes included adhesion and altered cell shape. No change in UMP-CMP kinase expression was detected, however, when assayed by RT-PCR. Alterations to incubation time or PMA concentration also failed to change UMP-CMP kinase expression. Similarly, no change was detected after DMSO treatment.

Because it was thought possible that CMP kinase activity might be required during rapid cell growth, the expression levels of the enzyme were assessed in various cancer cell lines. See FIG. 6B for the results of this investigation. K562 cells (human erythroleukemia cell line) expressed a slightly higher level of mRNA than other cancer cell lines tested. Other than this, all cancer cell lines assayed showed steady-state mRNA levels similar to those seen in normal tissues. In summary, without being limited to any one theory, CMP kinase appears to be ubiquitously present in all tissues and cell lines assayed. Expression does not appear to be developmentally regulated as monocytes differentiate into macrophages.

It was also considered that the 5′ region seen in the EST clone and not in the macrophage-derived clone might indicate that different transcripts are expressed in cancer cells than in normal cells. In order to investigate this possibility, primers were designed that hybridize to either the region not found in the macrophage-derived clone, or to a region common to both cDNAs. These primers were used in RT-PCR to determine that the 5′ region absent from the macrophage-derived clone is present in RNA expressed by both primary cultured cells (aortic smooth muscle cells) and cancer cell lines (HCT-116, RKO, Colo320, HCA7, LS174T). This suggests that the discrepancy between the macrophage-derived cDNA of the instant invention and the subsequently cloned cDNA of Van Rompay et al. does not result from RNA variants.

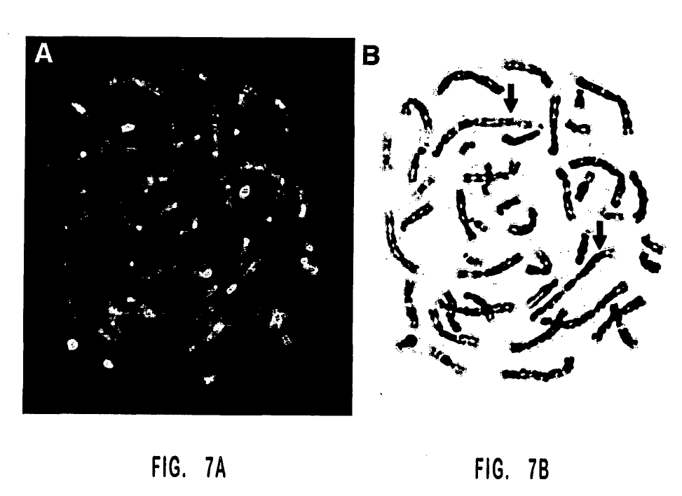

In a further effort to learn about this cDNA, its chromosomal location was determined. Using the UMP-CMP kinase cDNA as a probe, a genomic clone for this gene was obtained from a human genomic library (Bac). This clone was then used to assess the chromosomal localization of CMP kinase using fluorescence in situ hybridization (FISH). See FIG. 7A. The identity of the clone was confirmed by a Southern blot. The clone was then used to localize the UMP-CMP kinase gene to the short arm of chromosome 1 at region p34.1-32. The results of this experiment are shown in FIG. 7, in which arrows are used to indicate the physical location of CMP kinase. The band localization was confirmed on G-banded chromosomes. See FIG. 7B. Localization was confirmed on target DNA isolated from ten different cell lines. Interestingly, this location is a region that contains other nucleoside modifying enzymes.

While CMP kinase is clearly a novel enzyme, it exhibits striking similarity to AK-1. Both CMP kinase and AK-1 ubiquitously express two mRNA species and require Mg²⁺ for activity. The protein motifs shown to be important for catalysis in AK-1, as well as those shown to be important in pig UMP-CMP kinase, E. coli CMP kinase and other NMP kinases (Bucurenci et al., J. Biol. Chem., 271:2856-2862 (1996)) are highly conserved in this human UMP-CMP kinase. Given the importance of proper adenylate kinase function for normal erythrocyte stricture and function, it is likely that correct UMP-CMP kinase activity is also required. Indeed, one of the biochemical results of hyperactive pyrimidine nucleoside monophosphate (NMP) kinase was shown to be excess CDP accumulation in patients with hemolytic anemia. Lachant et al., 1986. Interestingly, downstream products of CDP, including CDP-ethanolamine and CDP-choline, were also elevated and may be the more direct cause of the erythrocyte phenotype.

This clearly defined importance of proper nucleotide levels suggests that altered UMP-CMP kinase activity will likely be linked to one or more disease processes. In contrast, the relatively modest ability of the enzyme to phosphorylate AMP suggests that this enzyme has a function entirely separate from that of AK-1 in the human cell. In contrast to this, the observation that ATP, in addition to UTP, is a favorable phosphate donor suggests at least some cooperation between UMP-CMP kinase and AK-1 in the task of maintaining proper relative nucleoside levels. These interpretations of the data obtained provide further support for the importance of proper CMP kinase activity and the likelihood that improper function would result in an abnormal phenotype in humans.

Hereditary ptosis (Engle et al., 1997) is the only disease known to be caused by a gene that lies within the same chromosomal region as the human UMP-CMP kinase. It is doubtful, however, that the condition is caused by improper nucleotide levels. Indeed, the seemingly ubiquitous nature of this UMP-CMP kinase suggests that it more likely performs housekeeping functions that may be required for viability. The fact that no CMP-kinase-deficient strain of E. coli is known to exist may indicate that such a mutation is lethal to this organi, and therefore possibly deleterious to humans. Fricke et al., 1995. In support of this conclusion, only conditional lethal mutants of E. coli dTMP kinase have been isolated and characterized Brinkely et al., 1986. Lack of developmental regulation from monocyte to macrophage formation further suggests that this gene must be actively translated from the beginning of the cell's developmental process.

While total lack of UMP-CMP kinase activity in humans is unlikely, the existence of families with AK-1 deficiency or PNMP kinase hyperactivity implies that aberrant UMP-CMP kinase could also be compatible with life. This also supports the hypothesis that such a condition would likely cause a significant physiological defect as do AK-1 and PNMP kinase abnormalities.

It is likely that some cancers will be included in the types of illnesses associated with improper CMP kinase activity. In support of this, a difference in PMNP kinase activity has been measured in rat hepatoma relative to normal rat liver. Maness and Orengo, 1976. Certainly, mutations occur during in vitro DNA synthesis when nucleotides are present in unequal proportions. Casson and Manser, 1995. It is therefore conceivable that improper CMP kinase activity could result in random mutations.

An even more likely function for UMP-CMP kinase is evident when considering nucleotide participation in membrane phospholipid synthesis. These types of bioactive lipids are involved in many biochemical pathways, as well as signaling pathways, that define the physical properties of biological membranes. If CMP kinase should alter reactions involving any of these important lipid mediators, many downstream events could be affected. In fact, deviations in the membrane lipid content could account for the malformed erythrocytes that occur with the aberrant PNMP kinase activity described above. As mentioned previously, CDP-ethanolamine and CDP-choline are elevated in erythrocytes isolated from PNMP kinase hyperactive patients. This is supported by the observation that deleted CMP kinase in E. coli resulted in perturbed membrane synthesis and a cold-sensitive phenotype.

Further insight into CMP kinase function was provided by the observation that the other genes involving nucleotide metabolism mapped to the short arm of chromosome 1, CTP synthase (1p34.1) and mitochondrial adenylate kinase-2 (1p34) are both near CMP kinase. Without being bound to any one theory, it appears likely that CMP kinase and CTP synthase act together to maintain proper CMP and CTP levels. This seems apparent because lowered CMP kinase activity in E. coli favors CTP synthase upregulation. Fricke et al., 1995. In addition, guanylate cyclase activator 2 also maps to chromosome 1 (1p35-p34). It is possible that these genes are part of a nucleotide metabolism gene cluster and that other similar enzymes will later be found to lie in this region of chromosome 1. The fact that E. coli CMP kinase has been found to be part of the rpsA operon which is required to maintain normal cell doubling time lends further credence to this theory. The discovery of additional nucleotide metabolizing enzymes on chromosome 1 may further explain the function of CMP kinase. In any case, this novel enzyme is highly likely to be involved in events other than the de novo nucleoside triphosphate synthesis pathways required simply for RNA and DNA synthesis. Its likely participation in membrane lipid composition places UMP-CMP kinase in a position to alter an endless number of cellular events.

All references, publications, patents, patent applications, and commercial materials cited in this application are hereby incorporated by reference in their entirety.

### 7. EXAMPLES

The following examples are given to illustrate various embodiments that have been made within the scope of the present invention. It is to be understood that the following examples are neither comprehensive nor exhaustive of the many types of embodiments that can be prepared in accordance with the present invention.

Experimental Methods:

cDNA Cloning and Sequencing

The UMP-CMP kinase cDNA was isolated from a human macrophage cDNA library. Tjoelker et al., Nature, 374: 549-552 (1995). Sequencing of both sense and antisense DNA was accomplished with the use of an ABI automated sequencer.

Transient Transfection

The UMP-CMP kinase cDNA was cloned into the BstXI sites of the mammalian expression vector pRc/CMV. HEK 293 or COS-7 cells were plated onto 6 well plates in Dulbelco Modified Essential Medium supplemented with 10% fetal bovine serum, penicillin (100 U/ml) and streptomycin (100 μg/ml). The following day, wells were transfected with 1 μg of either cDNA or vector only using 10 μl/well Lipofectamine (Glbco-BRL) according to manufacturer's instructions.

Western Blot

Sixteen hours after transfection, cells were washed once with PBS (pH 7.3) and scraped into 200 μl of lysis buffer (20 mM tris-HCl (pH 7.5), 1 mM EDTA, 16 mM CHAPS, 1 μg/ml leupeptin, 1 mM benzamidine, 10 μg/ml soybean trypsin inhibitor, and 0.5 mM dithiothreitol). The lysates were centrifuged (10,000×g, 10 min, 4° C.) and the supernatants removed for Western blot analysis. Proteins (50-100 μg/lane) were separated by SDS-PAGE (12% acrylamide) and transferred to Millipore Immobilon (PVDF) at 500 mA overnight. The membranes were blocked with 5% w/v nonfat dried milk in Tris-buffered saline (pH 7.3) with 0.1% Tween-20 for 1-2 hours at room temperature. UMP-CMP kinase protein was detected with rabbit anti-UMP-CMP kinase polyclonal antibody (diluted 1:250 in blocking buffer) raised against purified recombinant UMP-CMP kinase. The blots were then incubated with horseradish peroxidase conjugated goat anti-rabbit IgG secondary antibody (Kirkegaard and Perry) diluted 1:5000 in blocking buffer. Immunoreactive bands were visualized using Amersham's Enhanced Chemiluminescence (ECL) reagents according to the manufacturer's instructions.

Enzymatic Activity Assays

Cell lysates (25 μg total protein) prepared as described for Western blot were added to the following reaction mixture: 83 mM tris-HCl (pH 8.0), 3.3 mM MgCl₂, 0.17 M KCl, 6.7 mM NTP, 6.7 mM NMP and 0.1 μCi of the indicated [³H]-NU (60.4 mCi/mmol). The reaction was carried out at 37° C. for 6 min and terminated with an equal volume of 2 M formic acid. Unlabeled NDP was added (6.7 mM final concentration) and the reactions were dried under a steam of nitrogen. The products were resuspended in distilled H₂O and analyzed by thin layer chromatography.

Thin Layer Chromatography

Chromatography was performed by ascending development (dry start) in closed plexiglass chambers at room temperature using 0.25 M KH₂PO₄ (pH 3.4) as described previously. See Cashel et al., J. Chromotog., 40:103-109 (1969). The products were visualized under UV light and identified by co-migration with authentic nucleotides. Spots corresponding to different nucleotides were scraped from the plate and radioactive reaction products were quantified with a multi-purpose liquid scintillation counter.

Northern Blot

Human multi-tissue Northern blots (Clontech) were probed with UMP-CMP kinase cDNA. Specifically, the cDNA was excised, gel purified, and labeled using DIG DNA labeling kit (Boehringer/Mannheim). The membranes were prehybridized (30 min, 68° C. in Express Hyb (Clontech)) then hybridized to the UMP-CMP kinase probe (30 ng probe/mi Express Hyb, 1 hour, 68° C.). The membranes were washed (2×15 min, 0.1% SDS/2×SSC, room temperature, then 2×15 min, 0.1% SDS/0.2×SSC, 68° C.). The signal was detected with DIG nucleic acid detection kit and CSPD® (Boehringer/Mannheim).

Chromosomal Localization

A human genomic library (bacterial artificial chromosome) was screened with the macrophage library-derived UMP-CMP kinase cDNA (Genome Systems, Inc). Seven clones were isolated and subjected to a secondary screen, and then Southern blot analysis to confirm their identity. One clone was used for chromosomal localization by fluorescence in situ hybridization (FISH) analysis as previously described. See Pinkel et al., Proc. Natl. Acad Sci., USA 83, 2934-2938 (1986).

### Example 1

### 5′ Regions of UMP-CMP Kinase cDNAs: Identification of the Functional Translational Start Site

UMP-CMP kinase cDNA was cloned from a human macrophage cDNA library. The Basic Local Alignment Search Tool (BLAST) was utilized to compare the sequence of this clone with others submitted to the database. An expressed sequence tag (EST) clone also called human UMP-CMP kinase (AF070416) was identified through this process and determined to be derived from the same gene as the macrophage-derived UMP-CMP kinase cDNA. Van Rompay et al., Mol. Pharmacol., 56:562-569 (1999). This EST clone was, however, cloned after the macrophage-derived UMP-CMP kinase cDNA, and included an added 73 bases on the 5′ end not present in the macrophage-derived clone. See FIG. 8. Within this added sequence was an additional putative translational start site that would produce a 32 amino acid N-terminal extension of the expected protein product if the start site was functional.

In order to ascertain the existence of the N-terminal extension, the macrophage-derived UMP-CMP kinase cDNA was expressed in transfected cells, and the relative molecular mass of the protein product was determined by Western blot. The macrophage-derived cDNA produced a 25 kDa protein (FIG. 3A), the relative mass of which indicated that its sequence agreed with the predicted amino acid sequence (196 amino acids) of the gene product, and did not include the putative N-terminal peptide. The EST clone produced a protein of the same relative molecular mass when expressed. Van Rompay et al., Mol. Pharmacol., 56:562-569 (1999). Thus, both the macrophage-derived UMP-CMP kinase cDNA and the subsequently-cloned EST clone were shown to express proteins with the same relative molecular mass and appear to utilize the second start site, as illustrated in FIG. 8.

### Example 2

### Preferred Phosphate Acceptors for UMP-CMP Kinase Activity

It has been demonstrated that nucleotide kinases can often phosphorylate multiple types of nucleotides with varying relative kinetics. The report accompanying the EST clone indicated that the UMP-CMP case most efficiently phosphorylated CMP, UMP, and dCMP relative to other NMPs that were examined Van Rompay et al., Mol. Pharmacol., 56:562-569 (1999). In that initial study, it appeared that these three substrates were utilized with equal efficiency. Van Rompay et al., Mol. Pharnacol., 56:562-569 (1999).

In the research leading to the instant invention, it was confirmed that of those NMPs tested, the enzyme expressed by the macrophage-derived clone also prefers CMP and UMP as phosphate acceptors. FIG. 5B. Modest ATP phosphorylation above vector-transfected cells was also measured. FIG. 5A. These observations verify that the 5′ region absent from the macrophage-derived cDNA of this invention is not necessary to produce a catalytically active enzyme.

Contrary to the results accompanying the EST clone, however, the research involved in this invention demonstrated a distinct preference for CMP over UMP. Specifically, the product of the macrophage-derived UMP-CMP kinase cDNA was shown to cause a six- to seven-fold increase in CMP phosphorylation over the levels of CMP phosphorylation observed in vector-transfected cells. In contrst, this enzyme increased UMP phosphorylation by only twofold. FIG. 5A. This discrepancy in relative substrate preference could be caused by variations in the assay protocols utilized by the various research groups. These include differences in reaction time allowed (6 min. compared to 30 min.), and in the phosphate donor used (ATP compared to CTP).

### Example 3

### Preferred Phosphate Donors for UMP-CMP Kinase Activity

FIG. 5A illustrates the substrates shown by this research to be preferred by UMP-CMP kinase. In the research involving the instant invention, each NMP substrate was provided its corresponding NTP as a phosphate donor. In contrast, the evaluations conducted in Van Rompay et al. (1999) included ATP as the phosphate donor for all reactions regardless of the substrate. FIG. 5A.

The most preferred phosphate donor was investigated in order to more thoroughly define the optimal reaction conditions for UMP-CMP kinase activity. Because CMP was the most ideal phosphate acceptor found, it was included as substrate, while the NTP provided as a phosphate source was varied. It was determined that ATP and UTP are preferred over CTP and GTP as phosphate donors. FIG. 5B. It was shown that of these, CTP was least able to transfer phosphate to CMP, demonstrating approximately half the reaction efficiency of either ATP or UTP.

### Example 4

### Mg²⁺ Dependency of UMP-CMP Kinase Activity

Sequence comparison of the macrophagederived kinase with other nucleoside monophosphate (NMP) kinases revealed much similarity. This similarity was particularly notable within motifs known to confer functional properties. Among these similar regions was a Mg²⁺ binding loop. See FIG. 2. Bucurenci et al., J. Biol. Chem., 271:2856-2862 (1996). It therefore seemed reasonable that the cation requirements for UMP-CMP kinase might be similar to the requirements of other NMP kinases.

Through measurement of UMP-CMP activity in the presence of varying Mg²⁺ concentrations, it was demonstrated that Mg²⁺ increases UMP-CMP kinase activity in a concentration-dependent manner. FIG. 4. Maximal activity was observed in the presence of 3 mM Mg²⁺. The absence of Mg²⁺ brought CDP-formation down to the level observed in vector-transfected cells. It was thus demonstrated that UMP-CMP kinase activity has an absolute requirement for Mg²⁺. Without being bound to any particular theory, this appears to show that the putative Mg²⁺ binding loop in the predicted amino acid sequence is functional.

### Example 5

### UMP-CMP Kinase Expression Pattern

Van Rompay et al (1999) described UMP-CMP kinase steady-state mRNA levels in tissues from a variety of organ systems. The tissue distribution of UMP-CMP kinase was further investigated. Because the cDNA discussed in this application was cloned from a human macrophage library, expression in human immune tissues was assessed by Northern blot.

Two different mRNA species (3.4 and 2.0 kb) were shown to be expressed by all immune tissues examined. See FIG. 6A. The 3.4 kb form was the most prominent in every tissue on the blot. See FIG. 6A. Dual transcripts are likewise expressed by human adenylate kinase-1 (Matsuura et at, J. Biol. Chem. 264:10148-10155 (1989)), and it was presumed that UMP-CMP kinase also has splice variants. In contrast, only one transcript (approximately the size of the larger mRNA species shown in FIG. 6A) was detected on the more general multitissue Northern blot examined in the previous study. Van Rompay et al, Mol. Pharmacol., 56:562-569 (1999). No tissue-specific variation in expression levels was detected in immune tissues (FIG. 6A).

Due to the nature of the reaction catalyzed by UMP-CMP kinase, it was postulated that this enzyme is required during rapid cell growth. To test this, UMP-CMP kinase mRNA levels were assessed in various cancer cell lines. K562 (human erythroleukemia) cells were found to express a slightly higher level of mRNA (FIG. 6B), while other cancer cell lines had the low-level expression seen in the normal tissues. Both transcripts observed in the immune tissues were present in cancer cell lines.

It was also postulated that the presence of the added 5′ region in the EST clone and its absence in the macrophage-derived clone might serve to indicate that different transcripts are expressed in cancer cells than in normal cells. To test this, primers were designed that hybridize to either the region not present in the macrophage-derived clone or to a region common to both cDNAs. These primers were used to determine by RT-PCR that the 5′region absent from the macrophage-derived cDNA is present in RNA expressed by both primary cultured cells (aortic smooth muscle cells) and cancer cell lines (HCT-116, RKO, Colo320, HCA7, LS174T) (data not shown). This suggests that the discrepancy between the macrophage-derived cDNA and that from the EST described in Van Rompay et al. is not the result of RNA variants.

Because the cDNA had been cloned from a macrophage library, it was also considered possible that UMP-CMP kinase expression might be developmentally regulated as monocytes proceed toward the macrophage phenotype. To evaluate this possibility, Human monocytes were isolated and cultured for 10 days as they differentiated into macrophages. RT-PCR analysis of cells harvested on the day of plating and throughout the differentiation period showed no difference in UMP-CMP kinase mRNA levels (data not shown). In order to confirm this conclusion, HL-60 cells were employed. These cells are monocytic and can differentiate into macrophage-like cells in the presence of phorbol-12-myristate-13-acetate (PMA) (Murao et al., Cancer Res., 43:4989-4996 (1983), Rovera et al., Proc. Natl. Adac. Sci., USA 76:2779-2783 (1979), or into neutrophils in the presence of DMSO. Collins et al., Proc. Natl. Acad. Sci., USA 75:2458-2462 (1975). PMA clearly altered the HL-60 cell phenotype from a suspension cell to an adherent cell similar to isolated monocytes as they differentiate into macrophages. However, no change in UMP-CMP kinase expression was observed when assayed by RT-PCR This outcome was not changed by altering either incubation time or PMA dose. Similarly, no change in this message was detected after DMSO treatment (data not shown). These data indicate that UMP-CMP kinase is not developmentally expressed as monocytes differentiate. In conclusion, the ubiquitous nature of UMP-CMP kinase expression suggests that this enzyme is necessary for basic events common to all or most cell types.

### Example 6

### Chromosomal Localization

A genomic clone for UMP-CMP kinase was obtained using the macrophage-derived cDNA as a probe. This clone was used to localize the UMP-CMP kinase gene to chromosome 1 at region p34.1-32. See FIG. 7. This concurred with the chromosomal position reported by those who subsequently cloned the EST-based cDNA. (1p34.1-1p33). Van Rompay et al., Mol. Pharmacol., 56:562-569 (1999). It is interesting to note that this region of chromosome 1 contains other genes involved in nucleoside/nucleotide modification, including CTP synthetase (Yamauchi et al., Genomics 4:1088-1099 (1991)) and mitochondrial adenylate kinase-2 (Carritt et al., Ann. Hum. Genet 46:329335 (1982). Hereditary ptosis is the only known disease gene to colocalize with UMP-CMP kinase. Engle et al., Am. J. Hum. Genet. 60:1150-1157 (1997). However, no evidence exists linking this abnormality with improper nucleotide levels. Nevertheless, the ubiquitous expression of UMP-CMP kinase suggests that improper activity would result in appreciable illness, provided that it is not lethal. Future investigations will likely link aberrant UMP-CMP kinase activity to disease.

The invention may be embodied in other specific forms without departing from its essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. An isolated and purified nucleic acid molecule comprising a nucleotide sequence which encodes an amino acid sequence at least 70% identical to the amino acid sequence of SEQ ID NO: 2.

2. An isolated and purified nucleic acid molecule comprising a nucleotide sequence which encodes an amino acid sequence at least 80% identical to the amino acid sequence of SEQ ID NO: 2.

3. An isolated and purified nucleic acid molecule comprising a nucleotide sequence which encodes an amino acid sequence at least 90% identical to the amino acid sequence of SEQ ID NO: 2.

4. An isolated and purified nucleic acid molecule comprising a nucleotide sequence which encodes an amino acid sequence at least 95% identical to the amino acid sequence of SEQ ID NO: 2.

5. An isolated and purified nucleic acid molecule comprising a nucleotide sequence which encodes the amino acid sequence of SEQ ID NO: 2.

6. An isolated nucleic acid molecule which hybridizes to the complement of the nucleic acid molecule of SEQ ID NO: 1 when incubated for one hour (30 ng nucleic acid molecule/ml Express Hyb) at 68° C., followed by washing 2×15 min, 0.1% SDS/2×SSC at room temperature and then by washing 2×15 min, 0.1% SDS/0.2×SSC at 68° C.

7. The nucleic acid molecule of claim 6, wherein the nucleic acid molecule is subcloned into a plasmid.

8. The nucleic acid molecule of claim 6, wherein the nucleic acid molecule is subcloned into a prokaryotic or eukaryotic expression vector.

9. The nucleic acid molecule of claim 6, wherein the nucleic acid molecule is operably linked to a heterologous promoter.

10. The nucleic acid molecule of claim 6, wherein the nucleic acid molecule is stably or transiently incorporated into a prokaryotic or eukaryotic host cell.
